(19) United States
(12) Patent Application Publication
(10) Pub. No.: US 2014/0018780 A1
(43) Pub. Date: Jan. 16, 2014

(54) SURGICAL SHAFT-TYPE INSTRUMENT

(71) Applicant: Simon Hirscheld, Hamburg (DE)

(72) Inventor: Simon Hirscheld, Hamburg (DE)

(73) Assignee: OLYMPUS WINTER & IBE GMBH, Hamburg (DE)

(21) Appl. No.: 14/008,204

(22) PCT Filed: May 21, 2012
(86) PCT No.: PCT/EP12/02152

§ 371 Date: Sep. 27, 2013

(30) Foreign Application Priority Data
May 26, 2011 (DE) 10 2011 103 283.9

(51) Int. Cl. A61B 17/00

(52) U.S. Cl. CPC A61B 17/00
USPC 606/1

(57) ABSTRACT

A surgical shaft instrument has a shaft on whose distal end there is arranged a jaw mechanism having branches, at least one of which is movably mounted and on whose proximal end a handling mechanism is arranged, at least one handle part of which is movable for operation of the jaw mechanism, a transmission rod which is coupled at the distal end to the jaw mechanism and is coupled at the proximal end to the handling mechanism is provided and has a pulling element and a pushing element, such that the two elements are joined together at the ends, and one of the two elements is made of a superelastic alloy. The pushing element is made of the superelastic alloy and the pulling element is made of a non-superelastic material in the form of a cable or wire.

The present invention relates to a surgical shaft instrument of the type defined in the preamble of claim 1.

Surgical shaft instruments having a jaw mechanism on the distal end of an elongated shaft are frequently used in endoscopic procedures in a patient's body and are designed as forceps or scissors, depending on the design of the jaw. A transmitting rod ensures the transmission of pulling forces and compressive forces from a handling mechanism located at the proximal end to the jaw mechanism.

The use of superelastic alloys as disclosed in DE 43 13 903 C1 is also known for the transmitting rod. The actuator rod there is made of a superelastic material. In transmission of the closing force of the forceps by pulling on the actuator rod, the superelastic property is utilized to prevent transmission of excessively high closing forces and thus to prevent damage.

One disadvantage of this design, however, is that the actuator rod can be subject only to tensile loads. It would snap off under a compressive load.

The generic DE 199 08 593 A1 therefore designs the actuator rod to be comprised of a tensile element and a tubular pressure element, although the tubular pressure element may also be used for transmitting compressive forces, it is essentially used only to prevent buckling.

The use of a superelastic tensile element as in the two previously known designs can be utilized to limit the tensile force, i.e., for security to prevent damage and also for storing energy in forceps, which are clamped and then locked, as in those used as needle holders, for example.

However, the great superelastic extensibility of the tensile element also has disadvantages because it imparts an ambiguous actuating sensation, which thus stands in the way of accurate operation.

There is another problem that has not yet been discussed with shaft instruments, in which the shaft does not run in a straight line but instead is curved or bent, as shown in DE 10 2008 060 418 A1. In this case, the actuator rod must also run with a bend and/or kink and must still be displaceably operable beyond these break points. This results in slow-moving and inaccurate operability. This problem has so far been solved only unsatisfactorily with the known actuator rods.

DE 9404423 U1 describes forceps, for which it is reported that instead of a flexurally elastic and radially flexible metal tubing, a corresponding flexible tube, e.g., made of a superelastic nickel-titanium alloy, may also be used. Similarly, the flexible metal cable disclosed there can be replaced by a highly resilient rod and/or wire made of such a nickel-titanium alloy.

The object of the present invention is to create a generic surgical shaft instrument, which avoids an ambiguous actuation sensation while permitting accurate and smooth actuation, even when the shaft is bent or kinked.

This object is achieved with the features of the characterizing part of claim 1.

With the design according to the invention, the tensile element is made of a normal non-superelastic material of a low elasticity and can also transmit even higher forces very accurately. This tensile element is designed as a cable or wire and can thus be bent or curved very well to displace the bent or curved locations on a shaft without any loss of precision or mobility in operation. However, as is already known in the state of the art, this tensile element cannot be used for transmitting shearing because it would then buckle. For this purpose, the tubular pressure element is provided; because of its tubular shape, it can transmit shearing forces without any risk of snapping off. The resulting flexural rigidity, which would cause interference here, is greatly reduced according to the present invention due to the design of the pressure element being made of a superelastic material. The result is a transmission rod, whose two elements, namely the tensile element and the pressure element, can be bent well and/or can be kinked well. Nevertheless, the shearing effect can be transmitted over the pressure element. The great flexibility of the superelastic material does not cause much interference in the transmission of force because it is used only for the opening movement of the jaw in the case of forceps or scissors, and this requires only low forces and does not make any major demands regarding the accuracy of the transmission.

The tension element and the pressure element of the design according to the invention may be arranged side by side. Transmission of tension and pressure would also be possible here, in which case there will be a directional dependence, when used in a curved or kinked shaft tube. The features of Claim 2 are therefore advantageously provided. In the case of a concentric arrangement, the actuator rod can be bent equally well in all directions.

In the case of DE 199 02 593 A1, the tension element and the pressure element are fixedly connected to one another at one end. This presupposes a fixed connection between normal material and superelastic material, which entails major problems in production because of the extremely poor mechanical processability of superelastic materials. The features of claim 3 are therefore advantageously provided. The tension element is attached here to coupling parts at both ends. This does not pose any problems because the tension element is made of normal material. The pressure element, which is made of superelastic material, i.e., a material that is technically difficult to handle, is, however, arranged so it is only abutting at each end, which does not require any machining of the pressure element. The pressure element in the form of a tube, for example, may only have a straight end. Therefore, this eliminates the need for machining, e.g., to create a hole or a thread.

Other advantageous embodiments of the invention are derived from claims 4 through 8.

The drawing shows schematic diagrams illustrating the invention as an example.

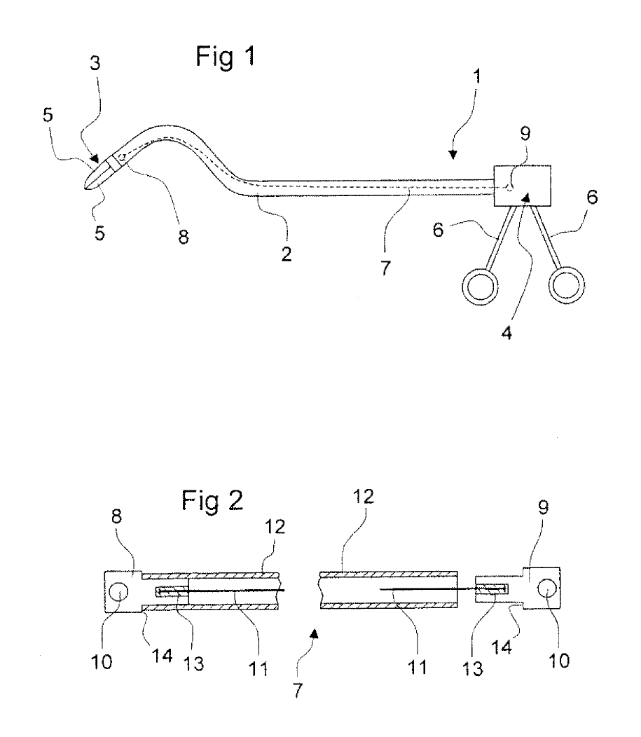

FIG. 1 shows a side view of a shaft instrument according to the invention with the transmission rod shown with a dotted line, and

FIG. 2 shows an enlarged and partially cut-away diagram of the transmission rod.

FIG. 1 shows a surgical shaft element 1 having an elongated tubular shaft 2 with a jaw mechanism 3 on its distal end having two jaw parts 5 that are movable toward one another.

The jaw mechanism 3 may be designed as tongs or scissors, depending on the design of the jaw parts 5. The jaw parts 5 may both be designed to be movable with respect to the shaft 2 or just one of the jaw parts may be movable while the other is arranged fixedly on the shaft 2.

On the proximal end of the shaft 2, a handling device 4 is arranged having two handle parts 6 which can be gripped with the hand, for example, in the finger rings in the example shown here, to move the handle parts 6, one or both of which may be designed to be movable, toward one another.

A transmission rod 7, which runs through the length of the shaft 2, is arranged in the interior of the tubular shaft 2. FIG. 1 shows the transmission rod 7 merely schematically with a dotted line, like the coupling parts 8 and 9 indicated schematically on the distal and proximal ends of the transmission rod 7. The coupling parts 8, 9 serve to provide operational coupling of the ends of the transmission rod 7 with the movement mechanism of the jaw mechanism 3 on the one hand and the handling device 4 on the other hand to transmit movements of the handle parts 6 for the purpose of moving the jaw parts 5.

FIG. 2 shows the transmission rod 7 in a preferred embodiment of the invention.

The coupling parts 8 and 9 which are already mentioned in conjunction with FIG. 1 are arranged on the ends of the transmission rod 7, each coupling part having a hole 10, which, in the exemplary embodiment shown here, serves the purpose of mechanical coupling with the moving parts of the jaw mechanism 3 on the one hand and the handling mechanism 4 on the other hand.

As shown in FIG. 2, the transmission rod 7 has a tension element 11 and a pressure element 12. The tension element 11 is embodied as a thin wire and/or a cable made of material that is not superelastic, i.e., made of normal elastic material, e.g., tool steel. The tension element 11 is attached to the coupling parts 8, 9 at each end. In the exemplary embodiment depicted here, the tension element is therefore provided with a thickened area on each of its ends and is cast in a borehole 13 in the coupling part 8 and/or 9. Any other suitable means of fastening may also be selected. For example, the tension element 11 may be crimped, welded, soldered or otherwise attached in the coupling parts 8, 9. No manufacturing problems occur here because both the tension element 11 and the coupling parts 8, 9 may be made of normal elastic material, for example, tool steel.

The tension element 11 may preferably be embodied as a multifilament cable which combines high strength with good flexibility. The tension element 11 may be made of metal. High-strength aramid and polyethylene fibers are especially suitable for this purpose.

Therefore, a connection having good tensile strength is established between the coupling parts 8 and 9, but this connection cannot transmit a shearing force because the bendable tension element 11 would then kink laterally immediately.

However, that is prevented by the pressure element 12, which is designed as a tube and can thus transmit shearing forces well in the direction of the axis of the tube without kinking. However, if the pressure element 12 were also made of a normal material having a normal elasticity, it would be very difficult to bend it or kink it laterally.

This would be a substantial disadvantage in using the transmission rod 7 in the shaft instrument 1 shown in FIG. 1, where the shaft 2 is bent, for example, kinked at two locations. As shown in FIG. 1, the transmission rod 7 must also be bent or kinked accordingly in these locations. Furthermore, the transmission rod 7 must be movable longitudinally in the shaft 2 in order to be able to execute transmitting movements. This all results in the requirement of very good and easy bendability of the transmission rod 7.

The transmission rod 7 illustrated in FIG. 2 has good bendability because of its design. The tension element 11 is then and thus also readily bendable in the embodiment in which it is made of normal material. The pressure element 12 is designed as a tube and does not bend easily but nevertheless has good lateral bendability because it is made of a superelastic material.

On the whole this yields a construction of the transmission rod 7, which can easily be longitudinally displaceable for actuation purposes in the arrangement according to FIG. 1, also at the bending and/or kink locations on the shaft 2.

As shown in FIG. 2, tensile forces are transmitted directly by the tension element 11 between the coupling parts 8 and 9. Shearing forces between the coupling parts 8, 9 are transmitted by the pressure element 12 with which only contact with the coupling parts 8, 9 in the pressure direction is required but a fixed connection is not required.

To prevent slackness, the coupling parts 8, 9 are preferably in contact with the ends of the pressure element 12 under prestress.

The design shown in FIG. 2 takes this into account. The coupling parts 8, 9 are each designed with a step 14 against which the respective end of the pressure element 12 can abut, such that the pressure element 12 is guided on a part of the coupling part 8 and/or 9.

This avoids manufacturing problems which would otherwise occur with a fastening that has both tensile and compressive strength, based on the superelastic properties of the pressure element 12.

FIG. 2 shows the two ends of the transmission rod 7 with the coupling part 8 on the distal end and the coupling part 9 on the proximal end. On the proximal end of the transmission rod 7, the pressure element is shown separately from the coupling part 9 for illustration, whereas on the distal end, the installation position is shown with a stop on the pressure element 12 against the step 14.

Since there is no fixed connection and in particular no closed connection between the ends of the tubular pressure element 12 and the coupling parts 8, 9, there is the possibility that dirt or microorganisms could enter the interior of the pressure element 12 and would be very difficult to reach there when cleaning and sterilizing the instrument for the purpose of reusing the surgical shaft instrument 1. To prevent dirt and microbes from entering the interior space of the pressure element 12, sealing measures may be provided in a manner not shown here, e.g., by means of elastic seals between the pressure element 12 and the coupling parts 8 and/or 9.

The term “superelastic alloy” used above is understood to refer to nickel-titanium alloys such as those known by the name “nitinol” which exhibits superelastic properties because of their special crystal structure and special thermal pretreatment.

1. A surgical shaft instrument comprising:
a shaft that includes:
a jaw mechanism arranged on a distal end of the shaft, the jaw mechanism having branches, at least one of the branches being mounted movably, and
a handling mechanism arranged on a proximal end, the handling mechanism having at least one handle part of which is movable for operation of the jaw mechanism; and
a transmission rod being coupled at the distal end to the jaw mechanism and coupled at the proximal end to the handling mechanism, the transmission rod having a pulling element and a pushing element, such that the pulling element and the pushing element are joined together at the ends, and one of the pulling element and the pushing element is made of a superelastic alloy, wherein the pushing element is made of the superelastic alloy and the pulling element is made of a non-superelastic material in the form of a cable or wire.

2. The surgical shaft instrument according to claim 1, wherein the pulling element is arranged concentrically in the pushing element.

3. The surgical shaft instrument according to claim 1, wherein the transmission rod has coupling parts designed on the ends for coupling with the jaw mechanism and/or with the handling mechanism, such that the pulling element is attached to each of the coupling parts and the pushing element is designed to abut in the pushing direction.

4. The surgical shaft instrument according to claim 1, wherein the pushing element is designed as a tube that is open on both ends, and the coupling parts, which are made of a non-superelastic material, are inserted into the ends thereof.

5. The surgical shaft instrument according to claim 4, wherein the pulling element is connected fixedly to the coupling parts.

6. The surgical shaft instrument according to claim 5, wherein the coupling parts are pulled toward the ends of the pushing element by the pulling element under prestress.

7. The surgical shaft instrument according to claim 1, wherein the pulling element is designed as a multifilament cable.

8. The surgical shaft instrument according to claim 7, wherein the pulling element is made of high-strength aramid or polyethylene fibers.
